(19) United States
(12) Patent Application Publication
(10) Pub. No.: US 2026/0308659 A1
(43) Pub. Date: Oct. 8, 2026

(54) CYCLIC BUILDUP OF BIOFILMS IN SMALL TO MEDIUM-SIZED LUMENS

(71) Applicant: Saban Ventures Pty Limited, Macquarie Park, NSW (AU)

(72) Inventors: Jonathan Graeme BURDACH, Lane Cove (AU); Tahria NAJNIN, Beaumont Hills, NSW (AU); Aleksandra Maria STANCZAK, Chiswick, NSW (AU); Blessy PATHROSE, St. Clair (AU); Kiranjit KAUR, Sydney (AU); Vaibhav Bipinchandra SHAH, Sydney (AU)

(21) Appl. No.: 19/480,600

(22) PCT Filed: Jun. 14, 2024
(86) PCT No.: PCT/IB2024/055864

§ 371 Date: Oct. 31, 2025

Related U.S. Application Data
(60) Provisional application No. 63/508,029, filed on Jun. 14, 2023.

(51) Int. Cl. C12Q 1/24; C12N 1/20; C12N 11/02

(52) U.S. Cl. CPC C12Q 1/24; C12N 1/20; C12N 11/02

(57) ABSTRACT

Provided are multi-cyclic methods for simulating the deposition of stable biofilms on the inner walls of small to medium inner diameter lumens found in medical devices such as endoscopes. Multiple embodiments, including four- and two-cycle versions of the cyclic build-up biofilm (CBB) model were developed for depositing tough and stable bacterial biofilms in the lumens having relatively small or medium sized internal diameters (≤2.5 mm ID). The resulting stable biofilms resemble the difficult to remove biofilms found in small lumened medical devices and provide a reliable model for simulating build-up that takes place over time in clinical settings. The CBB model also may be applicable to larger-lumened medical devices. Also provided are methods of cleaning such biofilms from lumens.

### CROSS-REFERENCE TO RELATED PATENT APPLICATIONS

This application claims the benefit of and priority to U.S. Provisional Patent Application Ser. No. 63/508,029, filed Jun. 14, 2023, which is incorporated herein by reference in its entirety.

### FIELD

This disclosure relates to a Cyclic Buildup Biofilm (CBB) model for simulating the deposition of a tough, stable biofilm in medical device lumens with small to medium internal diameters (ID), e.g., ≤about 2.5 mm. The method simulates deposition of multi-layered, stable biofilms frequently detected in endoscopes (e.g. gastroscopes) and other small-lumened devices following repeated cycles of clinical use and conventional cleaning and reprocessing techniques.

### BACKGROUND

Biofilms form when a group of bacterial cells senses a surface and begins to adhere to it. This is followed by colonization of more bacteria on the surface and the release of an extracellular polysaccharide matrix that solidifies the biofilm structure. The field of biofilm research is at a nascent stage but it already has been recognized that a biofilm is not simply a group of cells in a slimy, solidified structure but is encased in a highly ordered matrix that is vital to the survival of bacterial colonies of the biofilms. Biofilms formed by single or multiple species of bacteria are prevalent in nature, and multispecies biofilms in particular are complex, their structures dictated by environmental cues and inter-species interactions. The complexity of multispecies biofilms is underpinned by accumulating evidence suggesting that characteristics of multispecies biofilms cannot be deduced by observation of single-species biofilms.

To understand the role and composition of multispecies biofilm matrix and to design strategies to remove or limit the growth of biofilms, especially in medical devices, there is a need for biological models that simulate real-world conditions. Flexible endoscopes are associated with the greatest number of patient infections reported in the literature compared to any other reusable medical device [Rutala and Weber. Reprocessing semicritical items: outbreaks and current issues. Am. J. Infect. Control. 2019; 47: A79-89. doi.org/10.1016/j.ajic.2019.01.015]. Studies have demonstrated repeated transmission of the same strain from contaminated endoscopes to patients over time, highlighting the endoscope can be a long-term reservoir for infectious organisms despite reprocessing [Koveleva, et al. Is bacteriologic surveillance in endoscope reprocessing stringent enough? Endoscopy 2009; 41:913-16. doi.org/10.1055/s-0029-1215086; Rauwers, et al. Independent root-cause analysis of contributing factors, including dismantling of 2 duodenoscopes to investigate an outbreak of multidrug-resistant Klebsiella pneumoniae. Gastrointest. Endosc. 2019; 90:793-804. doi.org/10.1016/j.gie.2019.05.016]. In particular, flexible endoscopes and other lumened medical devices should not develop biofilms if they are adequately cleaned, disinfected and stored in dry conditions, that is, they are fully reprocessed. Inefficient or ineffective reprocessing, however, provides a conducive environment for the deposition and growth of biofilms. Recent evidence demonstrated that about 95% of in-use, flexible endoscopes still have visible water inside the channels despite undergoing a ‘dry cycle’ during reprocessing in an automated endoscope reprocessor [Ofstead, et al. The effectiveness of sterilization for flexible ureteroscopes: A real-world study. Am. J. Infect. Control. 2017 Aug. 1; 45 (8): 888-895]. Repeated cycles of exposure to patient secretions followed by compliant but inefficient and inadequate cleaning (reprocessing) and drying lead to a cyclic buildup of biofilms in flexible endoscopes, despite adherence to established instructions for use and clinical practice guidelines. [Primo, et al. Biofilm accumulation in new flexible gastroscope channels in clinical use. Infect Control Hosp Epidemiology 2022; 43:174-80. doi.org/10.1017/ice.2021.99.; Johani, et al. Determination of bacterial species present in biofilm contaminating the channels of clinical endoscopes. Infect. Dis. Heal. 2018; 23:189-96. doi.org/10.1016/j.idh.2018.06.003; Ren-Pei, et al. Correlation between the growth of bacterial biofilm in flexible endoscopes and endoscope reprocessing methods. Am. J. Infect. Control 2014; 42:1203-6. doi.org/10.1016/j.ajic.2014.07.029] Primo, et al. found biofilm accumulated in new gastroscope channels within 30-60 days of clinical use and reprocessing, most frequently in air/water channels [Primo, et al. 2022]. Evidence is also emerging about biofilm resistance to high-level disinfectants [Akinbobola, et al. ‘Secondary biofilms’ could cause failure of peracetic acid high-level disinfection of endoscopes. J. Hosp. Infect. 2021; 107:67-75. doi.org/10.1016/j.jhin.2020.09.028; Brunke, et al. Tolerance of biofilm of a carbapenem-resistant Klebsiella pneumoniae involved in a duodenoscopy-associated outbreak to the disinfectant used in reprocessing. Antimicrob. Resist. Infect. Control 2022; 11:81. doi.org/10.1186/s13756-022-01112-z]. Recently a clinical carbapenem-resistant Enterobacteriaceae (CRE) isolate from a duodenoscope-linked outbreak exhibited weak resistance against peracetic acid when in planktonic form, but strong resistance when in a biofilm form [Brunke, et al. 2022]. This study established a direct link between biofilm formation and high-level disinfection (HLD) tolerance in endoscopes to nosocomial transmission of CRE [Id.].

### SUMMARY

Provided are methods for simulating the buildup of a biofilm in lumened medical devices. Method embodiments involve building stable biofilms using a cyclic buildup biofilm (CBB) model in small- to medium-sized (≤about 2.5 mm) inner diameter lumens of medical devices. One embodiment includes preparation and filling a lumen of a media of at least 10⁶/mL CFUs of at least one species of bacteria, an artificial test soil, and blood, recirculating the media between about 20 and 30 degrees C. for at least four hours. This is followed by a rinsing and purging step, followed by filling the lumen with a fixing agent and rinsing and purging again. Optionally, the biofilm in the lumen may be stored at that point at least 5 to 6 hours or longer in a covered bin or under other conditions. In any given “cycle” the media may be recirculated at least four hours, but up to 40 or more, and the lumen rinsed and fluid purged before and after recirculation using any type of fluid and gas that may be suitable for such activities. In some embodiments the media is recirculated between about 16 and 20 hours at a flow rate of about 0.3 mL/min but both flow rates and recirculation times can be significantly increased.

Two- and four-cycle embodiments (over 24-hours per cycle) of the CBB model are highlighted but the number and length of cycles, as well as flow rates, reagents and other parameters may be varied depending on lumen size and the desired characteristics of the resulting biofilm.

In some embodiments the biofilm may be extracted from the lumen or from a part of the lumen for further testing. In certain embodiments the biofilm is extracted from the full length of the lumen. In other embodiments it is extracted using a flush-brush-flush method.

The CBB model may be applicable in larger lumened medical devices as well as several types of flexible endoscopes, for example, gastroscopes, bronchoscopes, duodenoscopes and colonoscopes.

### BRIEF DESCRIPTION OF DRAWINGS

FIGS. 1A-1D demonstrate results of evaluating timing and flow rate on biofilm deposition. FIG. 1A shows protein content, a proxy for biofilm deposition, as a function of time to deposit biofilm in larger (on left, ≥3.7 mm ID) versus smaller (medium-sized (on right, ≤about 2.5 mm ID) lumens. FIG. 1B shows protein content as a function of both time (2 versus 4 hours) and low (0.5 mL/min) versus high (1.5 mL/min) flow rates in small to medium-sized lumens. FIG. 1C shows protein content as a function of time (2 versus 18 hours) at a higher flow rate (1.6 mL/min) in small to medium-sized lumens; and FIG. 1D shows protein content as a function of two lower flow rates (0.5 mL/min versus 0.3 mL/min) in small to medium-sized lumens after 18 hours.

FIG. 2 shows the amount of protein and total organic carbon deposited in both 2.45- and 2.0-mm ID lumens, indicating that both of these small to medium-sized lumens collect nearly identical amounts of biofilm using the 4-cycle CBB model.

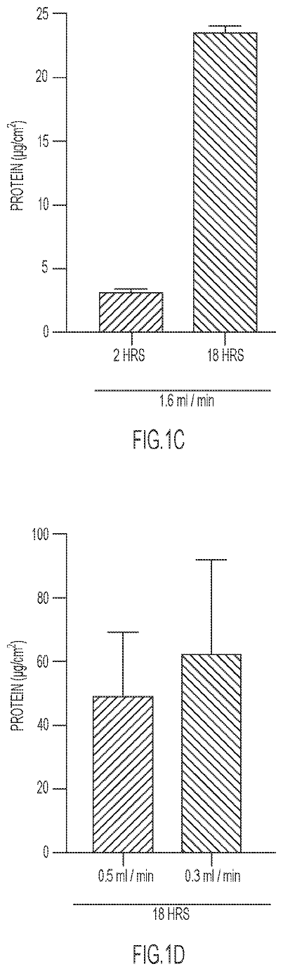

FIG. 3A is a photograph showing the required equipment assembled in the biosafety cabinet; FIG. 3B is a photograph showing the tubing and connectors used in the CBB model; and FIG. 3C is a photograph of the biofilm extraction process from small to medium-sized ID lumens carried out in a biosafety cabinet.

FIGS. 4A and 4B show two embodiments for the process of developing a stable biofilm using the Cyclic Buildup Biofilm (CBB) model in smaller or medium-sized ID (≤about 2.5 mm) lumens. FIG. 4A depicts a four-cycle CBB process that takes place over 4-5 days, including an extraction process, which may include extraction of the biofilm burden from the entire length of the lumen. FIG. 4B depicts an embodiment of a shorter, two-cycle CBB process that takes place over 2-3 days and including extraction. Separate embodiments for the extraction process are described in detail below.

FIG. 5 shows protein content (ug/cm²) remaining in the lumens of air/water and auxiliary (AW/AUX) channels of gastrointestinal endoscopes following cleaning using AECC (n=16) versus MC (n=16). An alert level of 3.0 μg/cm² from (ISO 15883-5:2021) was used as a benchmark for protein content. AECC is shown to leave significantly less protein—below the LOQ—in the lumens versus MC (p<0.001). Following MC, the amount of protein in the lumens remains significantly above the alert level (3.0 μg/cm²).

FIG. 6 shows TOC content (μg/cm²) remaining in the lumens of AW/AUX endoscope channels following AECC (n=16) versus MC (n=16) cleaning. An alert level of 6.0 μg/cm² from (ISO 15883-5:2021) was used as a benchmark for TOC content. AECC is shown to leave significantly less TOC remaining versus MC cleaning (p<0.001); further, following MC, the amount of TOC in the lumens remains significantly above the alert level (6.0 μg/cm²).

FIG. 7 shows the volume of bacteria as colony forming units (Log₁₀ CFU/cm²) of each of P. aeruginosa and E. faecalis remaining in the lumens of AW/AUX endoscope channels following AECC (n=16) versus MC (n=16) cleaning. AECC is shown to leave significantly fewer CFU of E. faecalis remaining (i.e., near the level of quantitation (LOQ) and P. aeruginosa is below the level of detection (LOD) in the lumens versus MC (p<0.001).

FIG. 8 is a graph showing the differences in total protein and total organic carbon (TOC) resulting from the four-cycle versus two-cycle CBB processes, indicating significantly lower (p<0.001) amounts of both TOC and protein (and thus, biofilm deposited) resulting from the shorter two-cycle CBB versus in the standard four-cycle CBB.

### DETAILED DESCRIPTION

This disclosure provides several embodiments of a Cyclic Buildup Biofilm (CBB) model, including a four-cycle, and shorter, two-cycle versions; each of the embodiments may be used for simulating the deposition of a tough, stable biofilm in small to medium-sized (≤about 2.5 mm) ID closed lumens of medical devices, such as flexible endoscopes or other medical devices. Multi-bacterial strain biofilms build-up over time in small to medium-sized endoscope lumens with cycles of use and reprocessing, despite close compliance with established cleaning standards and guidelines. Also disclosed are methods of evaluating the cleaning efficacy of different lumen cleaning techniques for lumens having small to medium-sized IDs.

The disclosed CBB model developed in smaller and medium-sized ID lumens simulates tough, stable biofilm development in endoscopes in clinical use wherein repeated cycles of exposure to patient secretions followed by cleaning, high-level disinfection (HLD), rinsing and drying leads to the deposition of biofilm: layers of organic material embedded with bacterial colonies, which are impervious to cleaning agents and disinfectants. The model involves more than a simple downsizing of the processes used in the past, which were developed for larger ID lumens of medical devices repeatedly exposed to bodily fluids. This disclosure also highlights the comparison of removal efficacy for such tough biofilms in these smaller ID lumens using an automated endoscope channel cleaner (AECC) versus a manual cleaning (MC) process in polytetrafluoroethylene (PTFE) surrogates with small or medium ID lumens used to simulate the cleaning process used for endoscope channels or channels of other small- or medium-lumened medical devices.

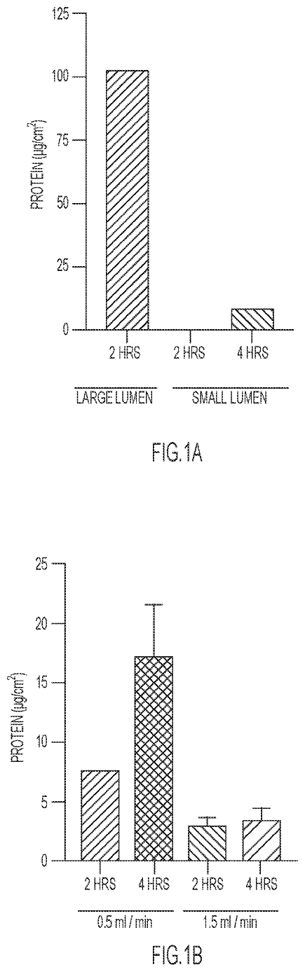

Efforts to replicate in lumens having smaller and medium-sized IDs the work previously carried out in larger lumens using a conventional technique did not result in a similar stable biofilm. Conventional methods yielded a significantly lower protein accumulation (a proxy for biofilm content) in smaller and medium-sized lumens after 2 and 4 hours using the same flow rate (1.25 ml/min) as used in larger lumens (FIG. 1A).

Growing evidence shows that individual bacteria species respond to mechanical stressors, including shear stress. The diversity of species in multispecies biofilms is influenced by changes in shear stress [Battin, et al. Effects of current velocity on the nascent architecture of stream microbial biofilms. Appl. Environ. Microbiol. 2003 September; 69 (9): 5443-52]. At a given flow rate, shear stresses imposed on the walls of smaller and medium-sized ID lumens will be different than in larger diameter lumens, potentially resulting in unstable deposition of biofilm arising from shorter recirculation time or changes in the composition of dual-species biofilm. Flow rate and recirculation time were optimized in the development of the CBB model to mitigate the effect of excessive shear stress in smaller and medium-sized lumens when replicating conditions for biofilm formation in larger lumens. Aspects presented herein include attempts to deposit biofilms at lower flow rates (e.g., 0.5 ml/min) and, alternatively, higher flow rates (e.g., 1.5 ml/min) than used previously at two and four hours. Doubling the recirculation time improved biofilm formation at a lower flow rate but not at a higher flow rate (FIG. 1B). Similar observations were made when the recirculation time was increased to 6 hours (data not shown). Recirculation time was increased at a faster flow rate (1.6 ml/min), demonstrating enhanced biofilm deposition (FIG. 1C). Finally, it was observed that increased recirculation time in conjunction with slower flow rates (0.3 ml/min and 0.5 ml/min) led to a stable and reproducible deposition of biofilm (FIG. 1D). Taken together, the data suggest that increased recirculation time improves the efficiency of biofilm formation regardless of the flow rate used during recirculation; lower flow rates are further beneficial for the deposition of biofilms in smaller and medium sized lumens. The data described in FIGS. 1A-1D were used to develop the CBB model for stable biofilm deposition in small and medium-sized lumens of medical devices, such as flexible endoscopes.

The disclosed CBB model (including the four-cycle and two-cycle embodiments) thus employs low flow rates (0.3 to about 0.5 ml/min) and extended recirculation times (16-20 hours) for smaller and medium-sized lumens.

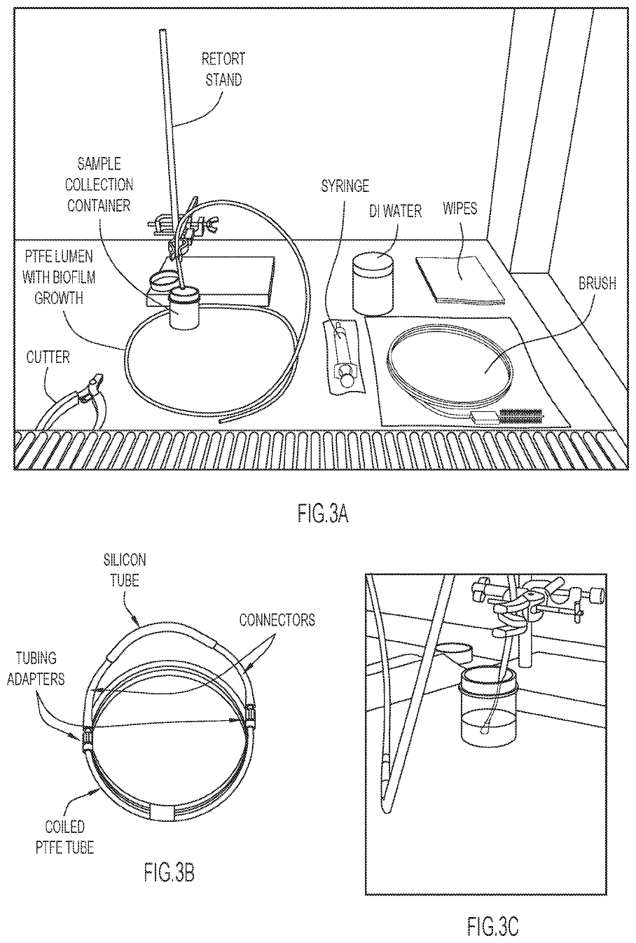

The four-cycle embodiment of the CBB model may be used for depositing a biofilm in a small and medium-sized ID lumen, typically over about four to about five days. In some embodiments using the shortened two-cycle CBB embodiment, the biofilm is deposited in about one to about two days, or about two to about three days; in still other embodiments the biofilm is deposited in about three to about four days; in other embodiments the biofilm is deposited in about four to about five days; in still other embodiments numerous additional “cycles” of biofilm deposition using this model may be required depending on the desired characteristics of the biofilm and require an additional number of days to execute each of the cycles. For example, the time required to deposit a stable biofilm using the disclosed CBB model may depend on the choice of particular reagents as well as the particular bacterial species selected, the type and size(s) of the lumens used, or other variable parameters. It should be appreciated that although the four-cycle and two-cycle processes (versions of the disclosed CBB model) are described herein at length, the embodiments of this disclosure encompass any number of additional “cycles” that may be added to the four-cycle embodiment. That is, the number of cycles, i.e., a series of steps in the four-cycle embodiment, (See e.g., FIG. 4A) may be increased as may be necessary to achieve a particular objective. For example, one may desire biofilms formed in lumens (i) of particular sizes and internal diameters, or (ii) having minimum or maximum biofilm (protein or total carbon) content. Others may include biofilms produced with a particular bacterial species or mix of species along with other characteristics or combinations of such characteristics. Thus, depending on the features of the lumens in which biofilms are desired (for testing, etc.), the precise conditions and reagents of the optional model embodiments, as well as the desired characteristics of the biofilms themselves, more than two, or more than four cycles may be required to achieve the desired results.

As a further example, in the case of a maximum level of biofilm desired, the number of cycles used in the disclosed model would be limited only by an indication that no additional biofilm could be deposited in a particular lumen; this could require 5 cycles, 6 cycles or even 20 or more cycles depending on the size of the lumen, species of bacteria, flow rates for recirculation, etc.

While the four-cycle and two-cycle embodiments of the disclosed CBB model were developed specifically for use in smaller and medium-sized lumens, some embodiments of the model also may be applicable for use in larger lumens, although, as noted above, the number of cycles may need to be increased to achieve a pre-determined biofilm build-up endpoint, which is a particular desired endpoint to deposition of a biofilm. The pre-determined biofilm build-up endpoint may include, for example, biofilm content as determined through one or more proxies such as total protein or total organic carbon (TOC), and/or other measures of a biofilm feature of interest within a particular size lumen, etc.

Accordingly, in some embodiments the number of additional cycles of the primary steps (at least steps 1 to 3 in FIGS. 4A and 4B) in the CBB model added to the four-cycle embodiment of the disclosed CBB model may range from 1 to 2, 2 to 3, 3 to 4, 4 to 5, 5 to 7, 8 to 10, 10 to 15, 15 to 20, 20 to 25, or 25 to 30 or more, depending on any number of parameters of the equipment, reagents, bacterial species or desired biofilm characteristics such as those described above. In some embodiments step 4 (storage at room temperature) as shown in FIGS. 4A and 4B, may be included in each cycle, or it may be omitted or varied in length or using other storage conditions depending on the intended use of the biofilm. Thus, in some embodiments, the primary Steps 1-3 as shown in FIGS. 4A and 4B constitute a cycle, without storage Step 4, while in other embodiments, Step 4 (as shown) is a component of a cycle.

As noted, storage of the biofilm within the lumen (as shown in the embodiment of FIGS. 4A and 4B, Step 4, and as Step 12 in the embodiment described in greater detail in the example below) may be omitted entirely. In some embodiments, as shown in FIGS. 4A and 4B, the lumen containing biofilm may be stored at room temperature in a covered bin about six hours to complete a typical 24-hour cycle, but this may be varied in other embodiments within a 24-hour cycle, or for any length of a “cycle,” particularly if the recirculation time is significantly modified. In still other embodiments, a skilled artisan will appreciate that the conditions of storage may be modified as requirements may dictate; for example, the ultimate, intended use of the biofilm may require that the lumen be stored under colder conditions, or for a much longer period of time.

Notably, the enhanced biofilm deposition provided by the disclosed CBB model can enable more accurate testing of reprocessing techniques. In other words, sufficiently high protein concentrations provided great enough resolution to test the efficiency of approaches to prevent the formation of biofilms, or to remove preformed biofilms. Although 16-20 hours of recirculation time was sufficient to produce stable biofilms with the CBB model in small to medium-sized ID lumens, 18 hours of recirculation resulted in the most effective and reproducible deposition of biofilms in lumens of a standard (1.8 m) length. Variations in the length and inner diameter, as well as other desired characteristics of the biofilms may cause the effective recirculation time for some embodiments to be reduced below 16 hours, or for other embodiments, increased above 20 hours.

For biofilms deposited according to the disclosed CBB model that may be developed in lumens of a larger size, e.g. >2.5 mm or larger, media recirculation time may change depending on a pre-determined biofilm build-up endpoint, for example recirculation times could decrease to between about 4 to about 6 hours, between about 6 to about 8 hours, between about 8 to about 10 hours, between about 10 and about 12 hours, between about 12 and about 14 hours, or between about 14 and about 16 hours, or could increase above about 20 hours to between about 20 hours and about 22 hours, between about 22 hours and about 24 hours, between about 24 hours and about 26 hours, between about 26 hours and about 28 hours, between about 28 hours and about 30 hours, between about 30 hours and about 34 hours between about 34 hours and about 40 hours or more in each cycle. Recirculation times greater than about 20 hours will alter the length of most 24-cycles, but the length of a cycle of the disclosed CBB model not limited to any set number of hours; for example, the embodiment shown in FIG. 4A is a 24-hour cycle including a storage period but in other embodiments, recirculation time alone could be extended well beyond 24 hours, suggesting that a cycle, with or without a storage period, could extend into two (or more) days.

The inner diameter of lumens used for building biofilms using the disclosed CBB process include the following: greater than about 5.0 mm, between about 5.0 and about 3.7 mm, between about 3.4 mm and about 3.7 mm, between about 3.1 mm and about 3.5 mm, between about 2.8 mm and about 3.3 mm, between about 2.5 mm and about 2.9 mm, between about 2.2 mm and about 2.6 mm, between about 1.9 mm and about 2.3 mm, between about 1.6 mm and about 2.0 mm, between about 1.3 mm and about 1.7 mm, between about 1.1 mm and about 1.5 mm, between about 0.9 mm and about 1.3 mm, between about 0.7 mm and about 1.1 mm, between about 0.6 mm and about 0.9 mm, between about 0.4 mm and about 0.7 mm, between about 0.2 mm and about 0.5 mm, and between about 0.1 and about 0.3 mm.

The flow rates used during the recirculation time using the four-cycle or two-cycle embodiments of the disclosed CBB model may range between about 0.1 mL/min to about 0.4 mL/min, between about 0.3 mL/min to about 0.5 mL/min, about 0.4 mL/min to about 0.6 mL/min, between about 0.5 mL/min to about 0.7 mL/min, between about 0.6 mL/min to about 0.9 mL/min, between about 0.8 mL/min to about 1.1. mL/min, between about 1.0 mL/min to about 1.2 mL/min, between about 1.1 mL/min to about 1.4 mL/min, between about 1.3 mL/min to about 1.6 mL/min, between about 1.5 mL/min to about 1.9 mL/min, and greater than about 2.2 mL/min, with flow rates greater than about 0.5 mL/min possible for use of the model in larger lumens.

Media recirculation temperature may range from between about 20° C. to about 30° C.; in other embodiments the temperature is set at between about 20° C. and about 23° C.; in still other embodiments the temperature is set at between about 24° C. and about 26° C., or between about 27° C. and about 30° C. In certain embodiments, the temperature is set at 25° C.

The number of colony forming units (CFU) that is included in the media that is prepared and recirculated in the lumens using the disclosed CBB model may vary depending on the amount of biofilm to be deposited. For example the concentration of CFU in the media can vary between about 10⁵/mL, 10⁶/mL, 10⁷/mL, 10⁸/mL, 10⁹/mL, and 10¹⁰/mL or more, in a 100 mL solution.

The length of the lumens used in the CBB model are 1.8 m, the length of a typical gastroscope channel, but may be shorter (about 1 m) or longer (e.g., 3 m), and with these modifications, appropriate adjustments may be made in the volumes of water, other liquids or air used in the process as described in detail in Example 2 and shown in the four-cycle and two-cycle embodiments, respectively, in FIGS. 4A and 4B.

The types of endoscopes that may be used with the CBB model include gastroscopes, colonoscopes, duodenoscope, and bronchoscopes, but other small- and large-lumened medical devices may also be applicable with this model.

The volume and velocity of a fluid (i.e., any type of suitable fluid such as water, any type of buffer or bacterial culture media, etc.) used for rinsing the lumens before fixation with a fixing agent affected the preformed biofilm, as underpinned by a recent publication demonstrating the sloughing of biofilm under certain shear dynamics Accordingly, the rinsing conditions have been optimized to avoid loss of preformed biofilm. Rinsing the lumens with, e.g., 10 mL to 20 mL of water, was sufficient to remove spent soil from the lumens, while rinsing with excessive amounts of water had a detrimental effect on biofilm deposition, which was exacerbated when a higher flow rate was used. As can be appreciated by one of ordinary skill in the art, more rinse water can be used for larger diameter contexts, but caution should be used as to both volume and flow rate to avoid dislodging any of the deposited biofilm.

Rinsing using any type of suitable fluid as mentioned above, and purging (using any type of suitable gas, e.g., air) of these smaller and medium-sized lumens takes place using a syringe (by hand) or with a peristaltic pump, and can vary significantly. For a typical 1.8 m lumen used in a gastroscope, this may be in the range of 10 to 13 mL/min, but that range could be higher or lower depending on changes to other dimensions of the lumen, as well as the method employed for rinsing: whether manual (via syringe) or some type of peristaltic pump. Using a peristaltic pump, in fact, would permit much lower flow rates for rinsing, e.g., to 0.3 mL/min or lower. In any event the rinsing flow rate should not be so high as to disturb the biofilm as the deposit is growing.

The disclosed CBB four- and two-cycle embodiments of the CBB model can be employed to study the characteristics of single- or multispecies biofilms and to evaluate cleaning efficiencies using various approaches. By altering the composition of one or more microorganisms used to contaminate the lumens, the model can be extended to simulate clinical biofilm deposition in various types of flexible endoscopes, in other small- and medium-lumened medical devices, and in most or all lumened medical devices with lumen internal diameters greater than 2.5 mm.

The two bacterial species used in the development of the CBB model for small lumens are Pseudomonas aeruginosa and Enterococcus faecalis, however, a number of bacterial species have been found in gastroscopes and colonoscopes, including but not limited to those in the following genera: Methylobacterium, Sphingomonas and Staphylococcus, Pseudommonas, Streptococcus, Shigella, Enterbacter and Enterococcus, and Mycolicibacterium. Without regard to the specific bacteria used to develop the CBB models, any one or more species of bacteria from any genus may be used in the disclosed method embodiments to form a stable biofilm under the CBB model using any of the disclosed embodiments.

The nuances associated with forming stable, single or multispecies biofilms will make working around the CBB model non-trivial to achieve desired results. More than merely introducing glutaraldehyde or another HLD would be required to replicate the disclosed model, since it accounts for differences between larger and smaller and medium-sized lumens in timing and content of biofilm deposition.

### Extraction of Biofilm

In some embodiments, the disclosed CBB model extracts the entire biofilm from the length of the lumen, e.g., typically 1.8 m for gastroscopes. In other embodiments, and depending on the purpose of the extraction, a portion of the lumen may be used for extraction, or a small sample of the biofilm may be extracted. In embodiments in which the entire lumen is used for extraction, in one embodiment, the lumen length is brushed and flushed to extract the biofilm, capturing the entire amount of biofilm in the lumen. A brush generates friction for efficient biofilm extraction. Extracting the entire biofilm burden from the lumen avoids the possibility of accidentally selecting sections of the lumen for extraction where the biofilm may not have grown evenly. The CBB model, thus, simulates extraction from contaminated flexible endoscopes in clinical settings. In other embodiments, other known extraction methods may be used to extract biofilm from the entire lumen, or a portion of it.

### Comparison of Cleaning Approaches: AECC Versus MC

Tough, stable biofilms grown under the CBB process over four or five days, were used to evaluate the efficacy of two cleaning methods for lumen with small to medium-sized inner diameters, such as those of AW/AUX channels. The Automated Endoscope Channel Cleaner (AECC) process was compared to a manual cleaner (MC) process using flushing and brushing. flushing.

Briefly as described in Example 2, the AECC process uses a specially formulated cleaning agent and delivery mechanism and an investigational device (CORIS, Nanosonics Ltd, Australia), according to the instructions for use. The process is disclosed in WO2022/256871A1, incorporated by reference in its entirety. Briefly, the cleaning agent was mixed with water in the device to create a saturated solution of both liquid and solid, which is perfused through the endoscope lumens at a high velocity to clean them. They are then flushed with water to remove the cleaning agent. An air purge follows to remove any residual liquid in the lumens, and then they are then dried through high level disinfection (HLD) or sterilization.

In the manual process, sterile deionized water is used to extract the biofilm content of narrow lumens using the flush-brush-flush method (i.e., 40 mL and 10 mL for 3.7 mm and 1.4 mm ID lumens, respectively). Samples are vortexed and sonicated and protein detection is performed using the bicinchoninic acid assay. Total organic carbon (TOC) detection was performed using the non-purgeable organic carbon method, and colony forming units (CFU) of the two bacterial species (P. aeruginosa and E. faecalis) were counted.

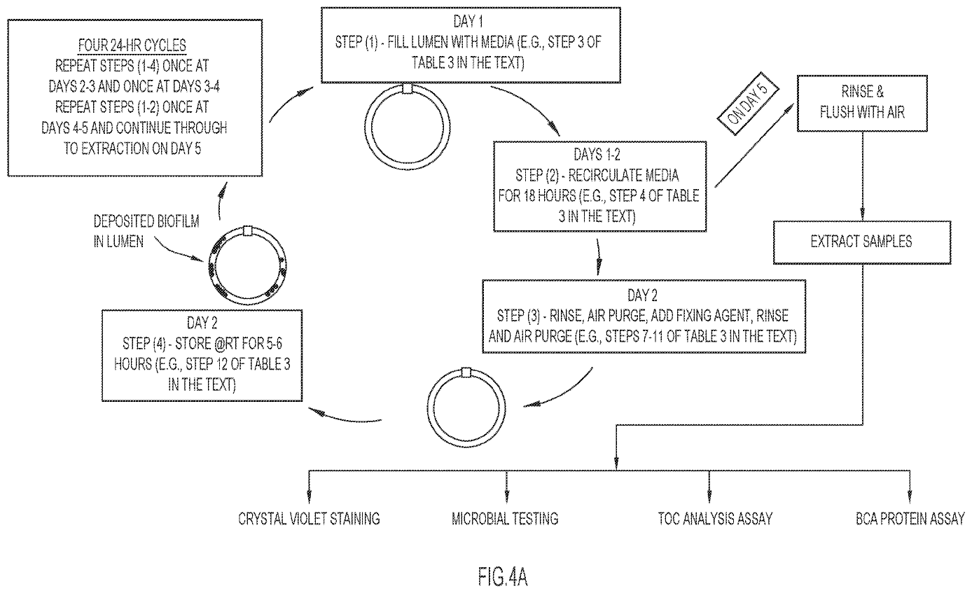

Results of the evaluation of cleaning processes are shown in FIGS. 5-7. The AECC significantly outperformed MC in removal of biofilm deposited using the disclosed CBB model in AW/AUX endoscope channels.

This disclosure describes an investigation of the stable biofilm deposited by the disclosed CBB model removal in 1.4 mm channels representing the AW/AUX channels of gastrointestinal endoscopes. In AW/AUX channels, MC failed to clean with little impact versus positive control. AECC reduced both protein and P. aeruginosa to below the LOD and TOC to below the alert level in these channels. MC was unable to remove CBB in AW/AUX channels with residuals remaining >10 times (protein) and >3 times (TOC) the alert levels. Over 10⁶ CFU/cm² of each organism also remained after MC. At these levels, HLD is unlikely to provide an adequate margin of safety, particularly as HLD may be compromised against organisms in biofilms [Akinbobola, et al. 2020; Brunke, et al. 2022]. The data shows standard MC practice is inadequate at removing CBB from narrow endoscope channels which cannot be brushed, providing probable root cause for a portion of the numerous endoscopy associated infections reported in the literature [Primo, et al. 2022; Johani, et al. 2018; Ren-Pei, et al. 2014; Akinbobola, et al. 2020; and Brunke, et al. 2022]. Primo et al. found that the majority of AW endoscope channels had extensive biofilm accumulation after only 30-60 days of patient-use and reprocessing cycles [Primo, et al. 2022], and Johani et al. found AW endoscope channels had a greater build-up of biofilm compared to working channels [Johani, et al. 2018]. The complexity of air/water (AW) channels including their varying diameter, attachment to valve seats and other structures, sharp bends and various joins further adds to the difficulty of achieving cleaning.

A limitation of the present disclosure is that MC was conducted in a laboratory with perfect adherence to the instructions for use. This does not reflect the case in reprocessing facilities where technicians are often under time pressure. Simulated endoscope channels were used to assess cleaning performance. Growing biofilm in isolated channels allows for proper extraction of residual cleaning markers. The inability to effectively sample narrow AW channels within endoscopes without destructive testing, is a known issue for biofilm recovery and limits biofilm detection clinically.

The present findings highlight the importance of using stringent, relevant biofilm models for evaluating cleaning efficacy for flexible endoscopes. ISO15883-5:2021 lists an example biofilm model that can be optionally used for flexible endoscope cleaning validation. This standard model involves continuously hydrated PTFE tubes inoculated with a single species (P. aeruginosa) in growth medium circulated for 3 to 4 days. The CBB model is a more stringent model (fixed, potentially multispecies biofilm grown in an organic test soil), developed to represent a challenge closer to that found in clinical practice. While not all endoscopes are exposed to glutaraldehyde fixation in clinical use, glutaraldehyde serves to create a worst-case simulated laboratory model in five days, whereas clinical biofilms can form over much longer periods. Continuously hydrated biofilms like the ISO15883 biofilm are morphologically different to fixed build-up biofilms like CBB, which yield compact layers of dried organic matrix with embedded organisms Use of the CBB model demonstrates a clear need for enhanced cleaning in channels that cannot be brushed. Selection of a relevant biofilm model in validation studies is of paramount importance for evaluation of new cleaning approaches.

The AECC cleans via the mechanical action of a cleaning agent passing through the lumens at high velocity. The ability to deliver physical cleaning of biofilm from all channels, regardless of diameter, should be considered a key indicator of cleaning success. Automation of cleaning can also eliminate the human factors associated with MC of endoscope channels.

Using the AECC method of cleaning the type of biofilms deposited in the small and medium-sized inner diameter lumens of endoscopes and other medical devices that is simulated by the CBB process, the amount of biofilm deposited can be reduced by at least about 99 percent, at least about 95%, at least about 90 percent, at least about 85 percent, at least about 80 percent, at least about 75 percent, at least about 70 percent, at least about 65 percent, at least about 60 percent, at least about 55 percent, at least about 50 percent, at least about 45 percent, at least about 40 percent, at least about 35 percent, at least about 30 percent, at least about 25 percent, at least about 20 percent, at least about 15 percent, at least about 10 percent, at least about 5 percent or at least about 1 percent.

Using the AECC method of cleaning the type of biofilms deposited in the small and medium-sized inner diameter lumens of endoscopes and other medical devices that is simulated by the CBB process, the amount of protein deposited can be reduced by at least about 99 percent, at least about 95%, at least about 90 percent, at least about 85 percent, at least about 80 percent, at least about 75 percent, at least about 70 percent, at least about 65 percent, at least about 60 percent, at least about 55 percent, at least about 50 percent, at least about 45 percent, at least about 40 percent, at least about 35 percent, at least about 30 percent, at least about 25 percent, at least about 20 percent, at least about 15 percent, at least about 10 percent, at least about 5 percent or at least about 1 percent.

Using the AECC method of cleaning the type of biofilms deposited in the small and medium-sized inner diameter lumens of endoscopes and other medical devices that is simulated by the CBB process, the amount of total organic carbon deposited can be reduced by at least about 99 percent, at least about 95%, at least about 90 percent, at least about 85 percent, at least about 80 percent, at least about 75 percent, at least about 70 percent, at least about 65 percent, at least about 60 percent, at least about 55 percent, at least about 50 percent, at least about 45 percent, at least about 40 percent, at least about 35 percent, at least about 30 percent, at least about 25 percent, at least about 20 percent, at least about 15 percent, at least about 10 percent, at least about 5 percent or at least about 1 percent.

Using the AECC method of cleaning the type of biofilms deposited in the small and medium-sized inner diameter lumens of endoscopes and other medical devices that is simulated by the CBB process, the number of colony forming units (CFUs) of any one or both of the bacterial species deposited can be reduced by at least about 99 percent, at least about 95%, at least about 90 percent, at least about 85 percent, at least about 80 percent, at least about 75 percent, at least about 70 percent, at least about 65 percent, at least about 60 percent, at least about 55 percent, at least about 50 percent, at least about 45 percent, at least about 40 percent, at least about 35 percent, at least about 30 percent, at least about 25 percent, at least about 20 percent, at least about 15 percent, at least about 10 percent, at least about 5 percent or at least about 1 percent. In certain embodiments CFUs are reduced by at least about 80 to 95 percent or more.

Accordingly, effective cleaning of endoscope channels is critical to ensure HLD or sterilization are effective and that endoscopes are safe for re-use. This disclosure shows that MC with strict adherence to instructions for use failed to remove biofilms deposited by the CBB process in 1.4 mm channels. Methods that deliver physical cleaning of biofilm across all channels are needed. The AECC tested cleaning of AW/AUX channels which are unable to be brushed. While further clinical studies are needed, AECC shows potential to deliver enhanced cleaning over current practice to small to medium-sized endoscope channels, and thereby addresses infection risk.

### Comparison of the Four-Cycle and Two-Cycle CBB Processes in Small and Medium-Sized ID Lumens

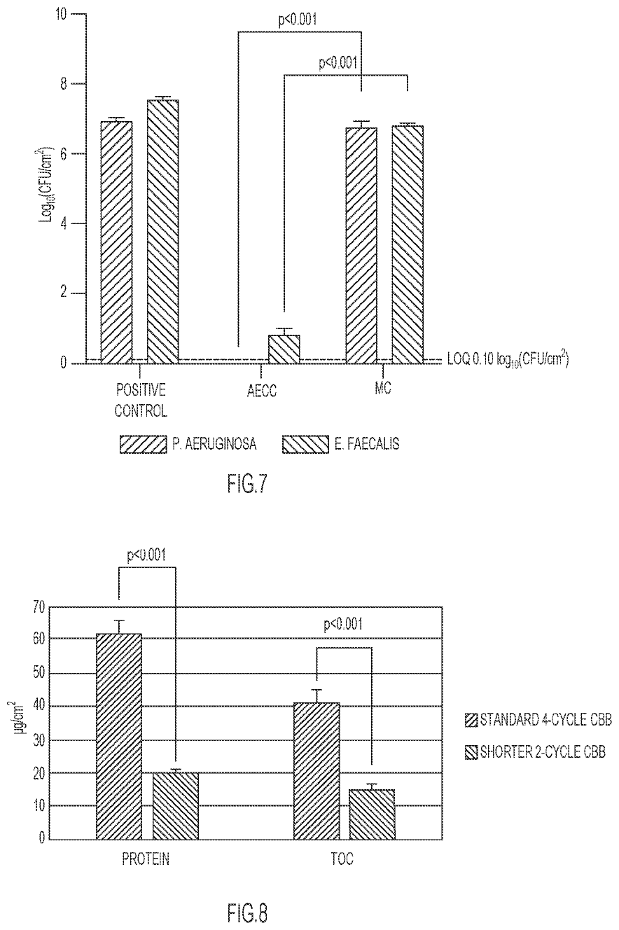

The four-cycle CBB process for small and medium-sized ID lumens was evaluated against an abbreviated two-cycle CBB process (both shown in FIGS. 4A and 4B) to determine biofilm content deposited in small and medium sized ID lumens. FIG. 8 shows a lower protein and TOC content deposited in the two-cycle process. Advantages of the two-cycle process include the quicker turn-around time and reduced use of reagents and technician time. Disadvantages include the fact that less biofilm is deposited with the two-cycle process and it is possible that the biofilm that is deposited in the two-cycle process (over approximately two to three days) may produce a weaker biofilm as compared to that which develops over four cycles (and over four to five days).

### Use of AECC for Cleaning Small and Medium-Sized ID Lumens of Endoscopes Following Clinical Use

In some method embodiments the AECC process is used for cleaning small and medium-sized ID lumens of endoscopes following clinical use to remove stable biofilms such as the type deposited using the CBB process over up to about five days. In one embodiment the biofilm comprises two bacterial species. In another embodiment, one of the bacterial species is P. aeruginosa, and the P. aeruginosa CFUs are reduced below the Level of Quantitation (LOQ) or below the Level of Detection (LOD). In yet another embodiment, one of the bacterial species is E. Faecalis and the E. faecalis CFUs are reduced below the LOQ through the use of the AECC process.

The Figures as provided herein illustrate exemplary methods and data. The order of the steps of the methods described herein also is exemplary, and unless otherwise specified, the steps may be carried out in any suitable order, or simultaneously where appropriate. Additionally, steps may be added or substituted in, or individual steps may be deleted from any of the methods without departing from the scope of the subject matter described herein. Embodiments disclosed herein may be combined to form further embodiments. Further, in some instances, not all acts may be required to enable implementation of a method described herein.

It will be understood that the above description of a preferred embodiment is given by way of example only and that various modifications may be made by those skilled in the art. What has been described above includes Examples of one or more embodiments. It is, of course, not possible to describe every conceivable modification and alteration of the above compounds, compositions or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

### EXAMPLES

### Example 1: CBB Model Biofilm Deposition in Small to Medium-Sized Inner Diameter (≤about 2.5 mm) Lumens, as Shown Generally in FIGS. 4A (Four Cycle CBB) and 4B (Two-Cycle CBB) Embodiments

### One Method Embodiment for Preparation of Bacterial Cultures and ATS2015 Media

Incubate Enterococcus faecalis (ATCC 29212) and Pseudomonas aeruginosa (ATCC 15442) working cultures (P2 to P6) in tryptone soya broth (TSB, CM0129, ThermoFisher) overnight at 37° C., and read absorbance at 600 nm (UV5 spectrophotometer, Mettler Toledo, Australia) to estimate colony forming unit (CFU) concentrations. CFU concentrations are calculated from the growth curves for each culture generated in our laboratory. The volume of ATS2015 media is prepared fresh just before use based on the guidelines in Table 1.

TABLE 1
Guide for preparation of ATS2015 Media based
on lumen ID of typical gastroscopes

| Inner lumen | | |
|---|---|---|
| diameter/ | ATS2015 media | Total ATS2015 media |
| lumen length | volume/day/lumen | volume needed |
| 2.45 mm/1.8 m | ~10 mL | 25 × no. of lumens = _{——}mL |
| 2.0 mm/1.8 m | ~8 mL | 8 × no. of lumens = _{——}mL |
| 1.4 mm/1.8 m | ~6 mL | 6 × no. of lumens = _{——}mL |

For example, to prepare 100 mL of ATS2015 media, weigh 9.5 μm of ATS2015 powder (ATS2015-250 mL, Healthmark Indust. Co.) in an empty sterile bottle and add 60 mL of sterile deionized water; mix by shaking with an orbital shaker for 10 minutes. Rest the solution for 20 minutes. Add 20 mL of defibrinated sheep blood (SD250D, Serum Australia) and swirl briefly. Mix a sufficient volume of each bacterial culture and sterile deionized water to achieve a final concentration of 1×10⁸ CFU/mL in 100 mL solution.

In other embodiments, other commercially available media including a mix of different species of bacteria, different numbers of species of bacteria or even a single species of bacteria may be used for this step.

### One Method Embodiment for Preparation of a Fixing Agent: Activated MetriCide (Active Ingredient-2.6% Glutaraldehyde)

The volume of Activated MetriCide (Metrex; SKU 10-1400) needed for the full experiment is prepared fresh just before use based on the guidelines in Table 2.

In other embodiments, any fixing agent with alkylated glutaraldehyde as the active ingredient may be used in the place of MetriCide. For example, high-level disinfectants with ortho-phthalaldehyde (OPA) can be used. Further, FDA-approved sterilants and high-level disinfectants such as peracetic acid and hydrogen peroxide also may be used to achieve similar levels of biofilm deposition using the four-cycle and two-cycle embodiments of the CBB model.

TABLE 2
Guide for preparation of activated disinfecting MetriCide
solution based on lumen ID size for certain embodiments

| | MetriCide | Metricide | |
|---|---|---|---|
| Lumen ID/ | volume/day/ | volume/lumen/ 4 | Total Volume |
| Length | lumen | days | needed |
| 2.45 mm/1.8 m | ~10 mL | ~40 mL | 40 × no. of |
| | | | lumens = _{——}mL |
| 2.0 mm/1.8 m | ~8 mL | ~32 mL | 32 × no. of |
| | | | lumens = _{——}mL |
| 1.4 mm/1.8 m | ~6 mL | ~25 mL | 25 × no. of |
| | | | lumens = _{——}mL |

Note that the length of the lumen may vary and, with it, so will the volumes/amounts presented here will vary in proportion to the change in the length of tubing.

Decant the required volume of MetriCide solution into a clean glass beaker and add the required amount of Activator 14 powder (supplied with the MetriCide) to the beaker (5.28 grams of powder/liter of MetriCide solution), stirring until it dissolves completely. The MetriCide solution is colorless but turns green upon dissolution of the Activator 14 powder, which raises the pH of the solution and activates the glutaraldehyde as a fixing agent.

The pH should be adjusted to 7.5-7.6 using a pH meter, adding more Activator 14 powder if necessary to achieve the desired pH.

Prior to each use during the course of the experiment, the amount of glutaraldehyde in the MetriCide solution should be tested using MetriTest™ test strips (Metrex; SKU 10-303) to ensure glutaraldehyde content remains above the minimum effective concentration (≥1.5% glutaraldehyde). The activated solution should be stored in an amber colored glass bottle for up to four days at room temperature, after which a fresh solution should be prepared.

### One Method Embodiment for Biofilm Growth Using the Four-Cycle CBB Model in Smaller and Medium-Sized Internal Diameter (ID) PTFE Lumens

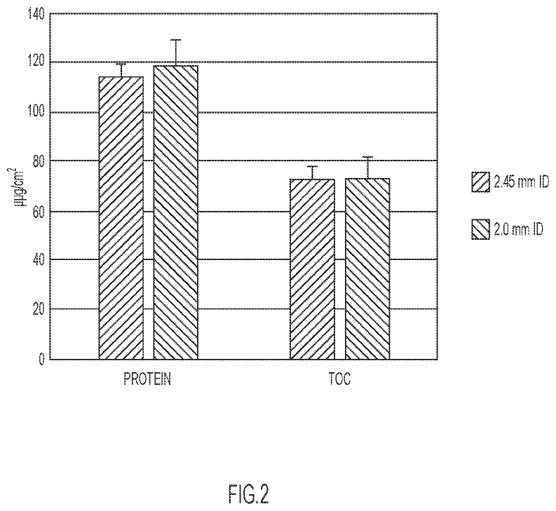

The equipment required for the CBB process is shown in FIG. 3A.

Inside a biosafety cabinet, connect two ends of a PTFE lumen via connectors attached to a sterile silicone tube to form a coiled lumen. To form a coiled 1.4 mm ID PTFE lumen, use tubing adaptors in addition to a silicone tube and connectors as shown in FIG. 3B.

The steps outlined below present one embodiment for growing biofilm in smaller and medium-sized ID PTFE lumens using the disclosed CBB model. The procedure below is written for 1.4 mm ID lumens, but it works for lumens of several smaller and medium-sized diameters (e.g., 1.51 mm, 2.0 mm, and 2.45 mm-all with an ID of up to about 2.5 mm) and, in certain other embodiments, may be applied in larger lumens (i.e., ≥3.7 mm or larger). Note that the CBB model's primary, ordered steps, as shown in the embodiments of FIGS. 4A and 4B as Steps 1-3, should not be reordered, substituted or omitted. Certain aspects of those steps of the CBB model, and the steps of the particular embodiment shown below—detailed amounts, reagents, and timing to a certain extent—may be modified in other embodiments without departing from the disclosed CBB model of depositing biofilm. For example, note that Step 12 below, which corresponds to Step 4 in FIGS. 4A and 4B (storage at room temperature), may be optional in some embodiments, as some biofilm will be deposited with each cycle of the primary Steps 1 through 3 in FIGS. 4A and 4B.

TABLE 3
Steps involved in growing cyclic-buildup biofilm
in 1.4 mm ID PTFE lumens in one embodiment

| | |
|---|---|
| Days 1-2 of | 1. Prepare MetriCide solution enough for Days 1-4 and store |
| 4-cycle CBB | appropriately. |
| protocol | 2. Prepare fresh ATS2015 Media. |
| Days 1-2 of | 3. Inside a biosafety cabinet, disconnect one end of the PTFE lumen |
| 2-cycle CBB | from the connector and completely fill the lumen with freshly |
| protocol | prepared ATS2015 Media using a sterile syringe. Push any trapped |
| | air bubbles out through the distal end of the lumen by injecting more |
| | ATS2015 Media. Reconnect the lumen end to its connector to close |
| | the loop again. |
| | 4. Set up a recirculation rig, attaching the 1.4 mm ID PTFE lumen to |
| | a pump and allowing ATS2015 Media to recirculate through the |
| | lumen at a flow rate of 0.3 mL/min at 25° C. for 16-20 hours. |
| Days 2-4 of | 5. Prepare fresh ATS2015 Media. |
| 4-cycle CBB | 6. After 18 hours of recirculation from previous day, remove the |
| protocol | lumen from the rig system, transfer to the biosafety cabinet. |
| Days 2-3 of | 7. Disconnect one end from the silicone connector and rinse with 10 |
| 2-cycle CBB | mL of sterile DI water using a sterile 20 mL syringe, collecting the |
| protocol | waste in an appropriate bottle. |
| | 8. Purge with 15 mL of air using the same 20 mL syringe. |
| | 9. Fill the lumen with 7 mL of activated MetriCide solution using a |
| | sterile 20 mL syringe and incubate for 20 min at room temperature. |
| | 10. Rinse with 40 mL (2 × 20 mL) of sterile DI water using a sterile |
| | 30-mL syringe, collecting the waste in an appropriate bottle. |
| | 11. Purge with 30 mL (2 × 15 mL) of air and connect the end of the |
| | PTFE lumen back to the silicone connector. |
| | 12. Store at room temperature for ~6 hours inside a basin with the lid |
| | on. |
| | 13. Inside a biosafety cabinet, disconnect one end of the PTFE lumen |
| | from the silicone connector and fill the lumen with freshly prepared |
| | ATS2015 Media, using a sterile syringe and reconnect. |
| | 14. Set up a recirculation rig, attaching the 1.4 mm ID PTFE lumen |
| | and allow ATS2015 Media to recirculate through the tubing at a flow |
| | rate of 0.3 ml/min at 25° C. for 16-20 hours. |
| Day 5 of 4- | 15. Repeat steps 6-8 as above. |
| cycle CBB | 16. Detach the silicone tubing with the connectors from the PTFE |
| protocol | lumen and rinse the lumen with 15 ml of sterile DI water using a |
| Day 3 of 2- | sterile 20 mL syringe. |
| cycle CBB | 17. Purge with 15 ml of air using the same 20 mL syringe. |
| protocol | 18. The lumen is ready for downstream processing. |
| (See FIGS. | |
| 4A and 4B) | |

The amount of water for rinsing and air for purging the lumen may vary in proportion with the lumen length. For example, for a lumen 1.8 m long, the amount of water used to rinse the lumen may vary between about 10 and about 50 mL, but this range may vary further depending on changes in length of the lumen; likewise, the amount of air used to purge the lumen following rinsing steps varies between about 15 and about 30 mL, again, varying with the length of the lumen but in any event is always sufficient to remove any residual liquid from the lumen before the next step in the process. A syringe may be used to rinse a lumen; alternatively, a peristaltic pump may be used, but in all cases care must be taken to rinse each lumen without forcing biofilm from the wall of the lumen in these pre-extraction stages. In general, to avoid disturbing the biofilm deposit as it grows, when rinsing with a syringe or peristaltic pump in a typical 1.8 m long lumen, the flow rate through the syringe can be in the 10 to about 13 mL/min range. Lumens of other dimensions in other embodiments may require higher or lower flow rates.

### One Method Embodiment for Extraction of CBB-Deposited Biofilm from Small and Medium-sized Inner Diameter, 1.8 m Long PTFE Lumens

One embodiment of the whole-lumen extraction process is shown below, but other embodiments may include the order of certain steps changed or reversed, or in other embodiments, certain steps omitted or modified slightly as to the quantities specified in the embodiment below.

In one embodiment, the whole lumen extraction process follows the following steps: A clean, sterile container is labeled for sample collection, and a PTFE lumen with biofilm growth is secured on a retort stand as shown in FIG. 3C. A suitable syringe is filled with half of the total extraction volume of sterile deionized water (5 mL).

The proximal end of a 200 μL micropipette tip is attached to the syringe and the distal end of the tip is attached as a connector into the lumen to inject sterile deionized water. The extracted liquid is collected on the other side of the lumen into the sample collection container.

An appropriately-sized brush (for small ID lumens, 1.8 m in length, e.g., Olympus brushes, BW-400B) is inserted into one end of the PTFE lumen and the entire lumen length is brushed; the brush is pulled back and the process of brushing is repeated three times.

After the last brushing step, the tip of the brush is snipped into the sample container.

Finally, the lumen is flushed with another half of the extraction volume (5 mL) of sterile deionized water and the extracted liquid (total, 10 mL) is collected into the same sample collection container.

The sample collection container is closed with a screw cap and it is stored at 4° C. until ready for analysis.

For analysis, the samples should be vortexed for 10 seconds, followed by sonication at 40 kHz for 5 minutes. Vortexing and sonication should be followed by a final 30-second vortexing. Note that this process including volumes specified may vary depending on the variation in length of the lumen.

The biofilm collected is then ready for downstream analysis, which may require, for example, histological (crystal violet) staining, microbial testing, BCA protein analysis, and total organic carbon (TOC) analysis or other analyses.

### Example 2: Comparison of Cleaning Procedures for Biofilm Produced Using the CBB Model of Biofilm Deposition

Biofilm growth. Autoclaved polytetraflurorethylene (PTFE) endoscope channels having lengths of 1.8 m, and internal diameters (IDs) of 1.4 mm, air/water and auxiliary (AW/AUX) channels (n=16), were used for preparing the improved CBB model biofilm. The CBB was grown with a circulation time of 18 hours for the bacteria-artificial test soil (ATS2015, Healthmark, Fraser, MI); storage time was 6 hours; flow rate was 0.3 ml/min; and there were three fixation cycles.

Cleaning procedure. Two cleaning procedures were used to remove biofilm deposited using the CBB model. The first type of cleaning process, a manual cleaner (MC) cleaning process was used to remove biofilm adhered to these small and medium-sized ID lumens (≤2.5 mm) found in flexible endoscopes (GIF/CF/PCF-190 Series, Olympus, Tokyo, Japan); the MC process used detergent (Endozime Xtreme Power, Ruhof, Mineola, NY), and instructions for use were adhered to for cleaning the 1.4 mm ID AW/AUX channels. In accordance with the instructions for use, brushing was not utilized in AW/AUX channels as they are inaccessible to brushes in endoscopes. The second cleaning process, automated endoscope channel cleaner (AECC) procedure with the investigational device (CORIS, Nanosonics Ltd, Australia) was performed per instructions for use for these same channels. AECC uses a specially formulated cleaning agent and delivery mechanism to physically clean all endoscope channels. The cleaning agent was mixed with water in the device to create a saturated solution of liquid and solid. Small quantities of this mixture were then propagated through the endoscope at high velocity to clean the channels. After cleaning, the channels were flushed with water and the non-toxic, water-soluble cleaning agent removed. An air purge then removed residual water. The endoscope was subsequently placed in an automated endoscope reprocessor for high level disinfection (HLD) or sterilization.

Sample Extraction. Sterile deionized water was used to extract channels using the brush-flush method (40 mL and 10 mL for 3.7 mm and 1.4 mm ID respectively). Samples were vortexed (10 seconds) and sonicated (40 kHz, 5 minutes) twice, then vortexed for 30 seconds.

Protein and Carbon Assays. Protein detection was performed using the bicinchoninic acid assay (Micro BCA protein assay 23235, Thermo Fisher Scientific, Waltham, MA). Total organic carbon (TOC) detection was performed using the non-purgeable organic carbon method (TOC-L CPH/CPN, Shimadzu, Kyoto, Japan). Pseudomonas aeruginosa (ATCC 15442) and Enterococcus faecalis (ATCC 29212) were detected by counting colony forming units (CFU) after incubation on trypticase soy agar (TSA) plates for 16-24 h). The limits for AW/AUX channels were as follows: protein assay LOQ=0.42 μg/cm² and LOD=0.13 μg/cm²; TOC assay LOQ=0.15 μg/cm² and LOD=0.05 μg/cm²; culture LOQ=1.26 CFU/cm² and LOD=0.13 CFU/cm².

Cleaning Benchmarks. Internationally recognized ISO 15883-5:2021 alert levels were used as cleaning benchmarks for protein (3 μg/cm²) and TOC (6 μg/cm²).

Statistical Analysis. All statistical analyses were conducted using Stata v18 (Stata Corp, College Station, Texas). The differences between cleaning conditions were estimated using linear regression. Statistical inference was calculated via the nonparametric bootstrap using 2000 bootstrap samples, due to non-normality of the distributions of residuals. Missing values for biofilm (Rᵢ), caused by actual values less than LOQ or less than LOD, were imputed by assigning random values either LOD<Rᵢ≤LOQ if below LOQ or 0≤Rᵢ≤LOD if below LOD. This procedure avoided artificial reduction of the within-condition variance and was repeated 1000 times and the study contrasts estimated within each resulting sample. The pooled results of the 1000 samples are reported. In effect, we utilized random processes to avoid any systematic bias in the estimated difference between cleaning conditions while also avoiding a potential artificial inflation of statistical power.

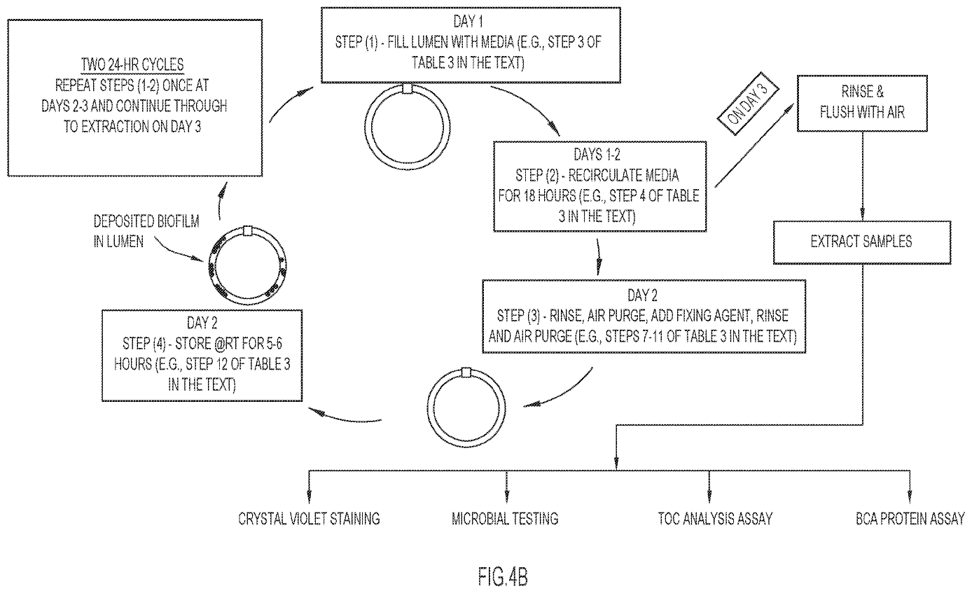

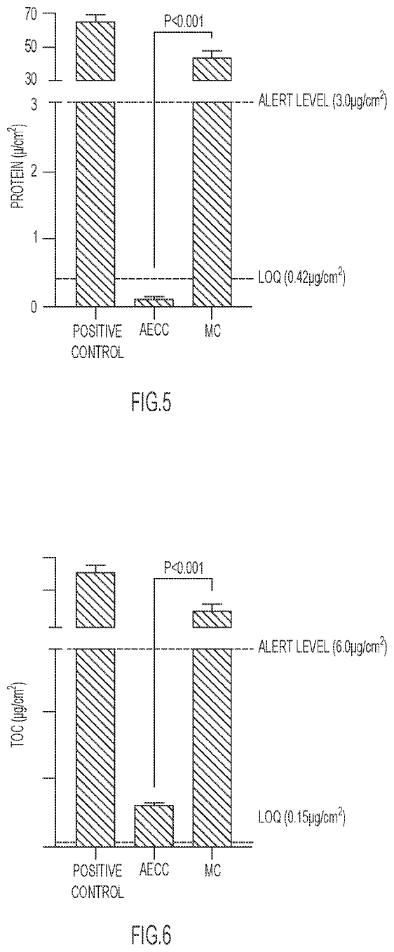

Results. With respect to the 1.4 mm ID AW/AUX channels, AECC showed a significantly improved capacity to remove both protein (FIG. 5, p<0.001) and TOC (FIG. 6, p<0.001) as compared to the MC process, and significantly reduced the number of CFUs of both bacterial strains (FIG. 7, p<0.001). The AECC process reduced P. aeruginosa to below the level of quantitation (LOQ).

### Example 3: Comparison of Protein and TOC Levels Resulting from the Four-Cycle Versus Two-Cycle CBB Process

Diagrams showing the four-cycle and the two-cycle embodiments of the CBB model for biofilm deposition are shown in FIGS. 4A and 4B and the steps for another embodiment are outlined briefly in Example 1. FIG. 8 shows the quantification of both protein and TOC in the four-cycle and two-cycle embodiments, showing a significantly reduced amount of protein (p<0.001) and TOC p<0.001) and, thus, total biofilm deposition, in the shortened two-cycle CBB process compared to the four-cycle CBB process. Evaluation of characteristics of the two-cycle biofilm layer deposition is ongoing.

1. A method for stable biofilm deposition using a cyclic buildup biofilm (CBB) method in a small- to medium-sized inner diameter lumen of a medical device, comprising the ordered steps of:
(a) preparing and filling the lumen with media comprising an artificial test soil, blood and a solution comprising a concentration of at least 10⁶ colony forming units (CFU) per mL (10⁶/mL) of at least one bacterial species;
(b) recirculating the media for at least 4 hours between 20° C. and 30° C.;
(c) rinsing the lumen with a fluid suitable for rinsing, purging any remaining fluid with a gas suitable for purging, filling the lumen with fixing agent, and repeating the rinsing and purging sufficiently to remove any fluid remaining in the lumen; and
(d) repeating a cycle of steps (a) through (c) in order at least once; and
(e) repeating steps (a) and (b) in order at least once,
wherein a stable biofilm is deposited in the small to medium-sized inner diameter lumen according to the CBB method, and
wherein the biofilm is comprised of multiple compact layers of dried organic matrix with embedded organisms.

2. (canceled)

3. The method of claim 1, wherein the recirculation temperature is 24° C. to 26° C.

4. (canceled)

5. The method of claim 1, wherein, following a last cycle of steps (a) through (c), the lumen is stored at room temperature.

6. The method of claim 5, wherein the lumen is stored for at least 5 to 6 hours.

7. The method of claim 1, wherein the medical device is selected from the group consisting of bronchoscope, duodenoscope, colonoscope and gastroscope.

8. The method of claim 1, wherein the medical device comprises an endoscope.

9. The method of claim 1, wherein the inner diameter of the lumen of the medical device is ≤about 2.5 mm.

10. (canceled)

11. The method of claim 1, wherein the inner diameter of the lumen of the medical device is between about 1.3 mm and about 1.7 mm.

12. (canceled)

13. The method of claim 1, wherein the fixing agent comprises glutaraldehyde and is activated to fix the biofilm layer inside the lumen when the fixing agent is adjusted to an alkaline pH.

14. The method of claim 1, wherein the fixing agent is selected from the group consisting of: alkylated glutaraldehyde, ortho-phthalaldehyde, peracetic acid, and hydrogen peroxide.

15-17. (canceled)

18. The method of claim 9, wherein any step in which the lumen is rinsed, an amount of the fluid used to rinse the lumen each time ranges from between about 10 mL to about 50 mL, and wherein an amount of the gas used to purge the fluid from the lumen each time ranges from between about 15 mL to 30 mL.

19. The method of claim 18, wherein the fluid used to rinse the lumen moves at a flow rate that ranges between about 10 mL/min to about 13 mL/min in an 1.8 m long lumen.

20-21. (canceled)

22. The method of claim 1, wherein, during step (b), the media recirculates at a flow rate that ranges from about 0.3 ml/min to about 0.5 ml/min.

23. (canceled)

24. The method of claim 1, wherein the amount of time the media recirculates is between about 4 and about 24 hours.

25-36. (canceled)

37. The method of claim 1, wherein the cycle of steps (a) through (c) is continued until a pre-defined biofilm buildup end point is reached, or until no further deposition of biofilm is possible or detectable in the lumen.

38. A method of extraction of the stable biofilm deposited in the lumen according to claim 1, the method comprising:
following a last repetition of steps (a) and (b), rinsing the lumen with the fluid and purging with the gas any remaining water in the lumen; and
removing the biofilm;
whereby the extracted biofilm is available for further analysis.

39. The method of extraction of claim 38, wherein the biofilm is removed from a full length of the lumen's inner walls.

40. The method of extraction of claim 39, wherein a flush-brush-flush process is employed to remove the biofilm from a full length of the lumen's inner walls.

41. The method of claim 1, wherein the fluid used to rinse the lumen is selected from the group consisting of water, a buffer, and a bacterial culture media.

42. (canceled)

43. The method of claim 1, wherein the gas used to purge the lumen of fluid is air.
